(19) United States
(12) Patent Application Publication
(10) Pub. No.: US 2026/0174712 A1
(43) Pub. Date: Jun. 25, 2026

(54) PHARMACEUTICAL COMPOSITION FOR PREVENTING OR TREATING RHEUMATOID ARTHRITIS COMPRISING PHENETHYL CINNAMAMIDE DERIVATIVE COMPOUND AS ACTIVE INGREDIENT

(71) Applicant: KNU-INDUSTRY COOPERATION FOUNDATION, Chuncheon-si (KR)

(72) Inventor: Koo Yeon LEE, Chuncheon-si (KR)

(73) Assignee: KNU-INDUSTRY COOPERATION FOUNDATION, Chuncheon-si (KR)

(21) Appl. No.: 19/411,668

(22) Filed: Dec. 8, 2025

(30) Foreign Application Priority Data
Dec. 19, 2024 (KR) 10-2024-0191637

(51) Int. Cl. A61K 31/165; A61K 36/185; A61P 19/02; C07C 235/34

(52) U.S. Cl. CPC A61K 31/165; A61K 36/3482; A61P 19/02; C07C 235/34

(57) ABSTRACT

The present disclosure relates to a pharmaceutical composition for preventing or treating rheumatoid arthritis, comprising a phenethyl cinnamide derivative compound as an active ingredient. The cannabinoid derivative compound may be effectively used in related industries by confirming effects of reducing an in vivo rheumatoid arthritis clinical index in a mouse model induced with rheumatoid arthritis, inhibiting damage to articular tissue, and reducing CII IgG antibody production in mouse serum.

### CROSS-REFERENCE TO RELATED APPLICATION

This application claims the priority of Korean Patent Application No. 10-2024-0191637 filed on Dec. 19, 2024, in the Korean Intellectual Property Office, the disclosure of which is incorporated herein by reference.

### BACKGROUND

### Field

The present disclosure relates to a pharmaceutical composition for preventing or treating rheumatoid arthritis including a phenethyl cinnamamide derivative compound as an active ingredient.

### Description of the Related Art

Autoimmune rheumatoid arthritis is a disease characterized by the synovial inflammation, neovascularization, and damage to articular cartilage and bone caused by an increase in inflammatory transmitters according to abnormal immune responses, and its prevalence has been currently known to be approximately 18. The autoimmune rheumatoid arthritis is an immune-mediated disease that causes abnormalities in the immune system due to genetic factors, and environmental factors such as infection, and a disease whose cause has not yet been known.

To elucidate the pathogenesis of the autoimmune rheumatoid arthritis, many researchers have conducted experiments on cells involved in a series of articular destruction processes. However, joints are not simply empty spaces between bones, but complex organ-like structures which are constituted by organically connecting various parts, such as synovial membranes, cartilage, bones, and bloods, and subtly influence each other through interactions between cells and molecules capable of inducing various immune responses, such as cytokines. Thus, there are many variables to be considered when treating arthritis, and thus it is difficult to be treated. As such, a pathogenic inflammatory response in autoimmune rheumatoid arthritis is a complex pathological disease, so that a multifaceted therapeutic approach is urgently required.

In addition, the autoimmune rheumatoid arthritis is also accompanied by osteoporosis and systemic organ invasion (lungs, skin, and eyes) as well as articular lesions to significantly reduce the quality of life of patients and makes normal life impossible to lead to social isolation of patients. The autoimmune rheumatoid arthritis, as a product of inappropriate immune function, is an incurable disease that is difficult to be diagnosed and treated for a long time, and becomes a factor causing serious social and economic problems. In such a situation, research should be conducted to control the fundamental cause based on complex pathological phenomena.

Meanwhile, Hemp (Cannabis sativa L.) is an annual plant of the genus Cannabis, the family Cannabaceae, which has been widely cultivated in tropical and temperate regions, mainly in Central Asia, since 12,000 years ago, and includes wild ginseng, and collectively refers to Cannabis chemovars and their variants, and Cannabis sativa subspecies sativa, Cannabis sativa subspecies indica, Cannabis sativa subspecies ruderalis, including varieties var. indica and var. kafiristanica, and their genetically cross-fertilized, self-fertilized or hybridized plants, containing various types of cannabinoid compounds known as medicinal and pharmaceutical component.

In traditional Chinese and Korean medicine books, Cannabis Semen, as hulled hemp seeds, has been used for constipation, diabetes, pain disorders, menstrual irregularities, skin diseases, and dysentery, and Cannabis sativa, as hemp leaves, has been used as anti-parasitic, hair protection, asthma treatment, analgesic, anesthetic, and diuretic agents. In addition, the hemp roots have been used for treatment of dystocia and relief of blood stasis, the hemp barks have been used for bruises and open wounds, the hemp flowers have been used for paralysis, itching, etc., and the hemp pollens have been used for dystocia, constipation, gout, insomnia, etc. As such, there are records showing that each part of the hemp was used according to symptoms.

The hemp contains approximately 400 compounds, most of the compounds include cannabinoids, terpenes, and phenolic compounds, and among these compounds, there are approximately 90 types of cannabinoids, which are important medicinal and pharmaceutical natural ingredients, and many ingredients thereof was found only in the hemp. Among the cannabinoids in the hemp, a psychoactive ingredient is Δ9-tetrahydrocannabinol (Δ9-THC), and cannabidiol (CBD) is a non-psychoactive ingredient and known as an ingredient which exhibits a physiologically active effect through various receptors in the human body, including an adrenergic receptor and a cannabinoid receptor.

Accordingly, the present inventors confirmed the effects of reducing the rheumatoid arthritis clinical index in a rheumatoid arthritis-induced mouse model, inhibiting damage to articular tissues, and reducing a CII IgG antibody production in the serum by using a novel hemp-derived phenethyl cinnamide derivative compound, and then completed the present disclosure.

### SUMMARY

An object of the present disclosure is to provide a compound represented by the following Chemical Formula 1 or a pharmaceutically acceptable salt thereof.

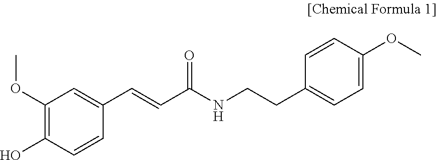 [SMILES: COc1ccc(CCNC(=O)/C=C/c2ccc(O)c(OC)c2)cc1]

Another object of the present disclosure is to provide a pharmaceutical composition for preventing or treating rheumatoid arthritis, including the compound represented by Chemical Formula 1 or the pharmaceutically acceptable salt thereof as an active ingredient.

Yet another object of the present disclosure is to provide a food composition for preventing or alleviating rheumatoid arthritis, including the compound represented by Chemical Formula 1 or the pharmaceutically acceptable salt thereof as an active ingredient.

In order to achieve the above object, an aspect of the present disclosure provides a compound represented by the following Chemical Formula 1 or a pharmaceutically acceptable salt thereof.

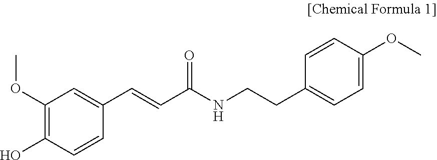 [SMILES: COc1ccc(CCNC(=O)/C=C/c2ccc(O)c(OC)c2)cc1]

Further, another aspect of the present disclosure provides a pharmaceutical composition for preventing or treating rheumatoid arthritis, including the compound represented by Chemical Formula 1 or the pharmaceutically acceptable salt thereof as an active ingredient.

Further, yet another aspect of the present disclosure provides a food composition for preventing or alleviating rheumatoid arthritis, including the compound represented by Chemical Formula 1 or the pharmaceutically acceptable salt thereof as an active ingredient.

According to the present disclosure, the novel phenethyl cinnamide derivative compound can be effectively used in related industries by confirming the effects of reducing the in vivo rheumatoid arthritis clinical index in a rheumatoid arthritis-induced mouse model, inhibiting damage to articular tissues, and reducing the CII IgG antibody production in the mouse serum.

### BRIEF DESCRIPTION OF THE DRAWING

The above and other aspects, features and other advantages of the present disclosure will be more clearly understood from the following detailed description taken in conjunction with the accompanying drawings, in which:

FIG. 1 shows cell viability of 14 types of compounds of the present disclosure;

FIG. 2 shows intracellular nitric oxide production of 14 types of compounds of the present disclosure;

FIG. 3 shows intracellular TNF-α expression levels of 14 types of compounds of the present disclosure;

FIG. 4 shows intracellular IL-6 expression levels of 14 types of compounds of the present disclosure;

FIG. 5 shows clinical scores for compounds of the present disclosure in a rheumatoid arthritis mouse model;

FIG. 6 shows weight changes for compounds of the present disclosure in a rheumatoid arthritis mouse model;

FIG. 7 shows spleen weight changes for compounds of the present disclosure in a rheumatoid arthritis mouse model;

FIG. 8 shows paw photographs for compounds of the present disclosure in a rheumatoid arthritis mouse model;

FIG. 9 shows H&E staining of paw tissues for compounds of the present disclosure in a rheumatoid arthritis mouse model; and

FIG. 10 shows CII-IgG antibody production in the serum for compounds of the present disclosure in a rheumatoid arthritis mouse model.

### DETAILED DESCRIPTION

Hereinafter, an example of the present disclosure will be described in detail with reference to the accompanying drawings. However, the following examples are presented as examples for the present disclosure, and when it is determined that the detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Further, terminologies used in the present disclosure are terminologies used to properly express examples of the present disclosure, which may vary according to a user, an operator's intention, or customs in the art to which the present disclosure pertains. Accordingly, definitions of the terminologies need to be described based on contents throughout this specification.

Throughout this specification, unless explicitly described to the contrary, when a certain part “comprises” a certain component, it will be meant to further comprise other components rather than excluding other components.

Hereinafter, terms used in the present disclosure will be described.

The present disclosure provides a compound represented by the following Chemical Formula 1 or a pharmaceutically acceptable salt thereof.

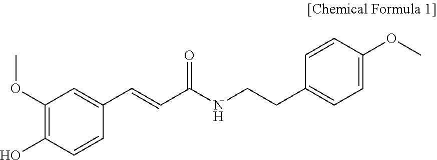 [SMILES: COc1ccc(CCNC(=O)/C=C/c2ccc(O)c(OC)c2)cc1]

The compound represented by Chemical Formula 1 above may be (2E)-3-(4-hydroxy-3-methoxyphenyl)-N-[2-(4-methoxyphenyl)ethyl]-2-propenamide (hereinafter referred to as “Compound 10” or “KMC-0245”).

The compound may be a phenethyl cinnamamide derivative.

The compound represented by Chemical Formula 1 of the present disclosure may be prepared using the following Reaction Scheme 10.

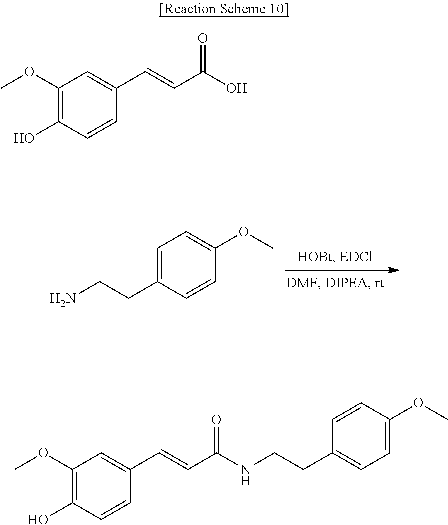

In the present disclosure, “pharmaceutically acceptable” means not significantly stimulating a living organism and not inhibiting the biological activity and properties of an administered active substance.

According to one embodiment of the present disclosure, the compound may be a Hemp-derived phenethyl cinnamamide derivative.

The “Hemp” of the present disclosure is a type of product derived from Cannabis sativa, and the Hemp has a very low concentration of THC (as a mind-altering ingredient) and does not cause hallucinations like marijuana, and thus may be used as industrial and health-related products.

According to one embodiment of the present disclosure, the compound may have cell viability of 80% or more.

According to one embodiment of the present disclosure, the compound may inhibit nitric oxide production.

According to one embodiment of the present disclosure, the compound may inhibit IL-6 expression.

Further, the present disclosure provides a pharmaceutical composition for preventing or treating rheumatoid arthritis, including the compound represented by Chemical Formula 1 or the pharmaceutically acceptable salt thereof as an active ingredient.

As used herein, the term “prevention” refers to all actions that inhibit the symptoms of a specific disease or delay its progression by administering the composition of the present disclosure.

As used herein, the term “treatment” refers to all actions that improve or beneficially change the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents, which are commonly used in a pharmaceutical field.

The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oils.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing the active ingredient with at least one or more excipients, such as starch, calcium carbonate, sucrose, lactose, gelatin, etc. Further, lubricants such as magnesium stearate and talc may be used in addition to simple excipients. Liquid formulations for oral administration may correspond to suspensions, oral liquids, emulsions, syrups, etc., and may include various excipients, such as a wetting agent, a sweetener, an aromatic agent, a preserving agent, etc., in addition to commonly used diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As a base material of the suppository, witepsol, Tween 61, cacao butter, laurinum, glycerogelatin, etc. may be used.

The pharmaceutical composition according to the present disclosure may be administered to a subject through various routes.

All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, physical conditions, and the like of a subject. It is obvious that the concentration of the active ingredient included in the pharmaceutical composition may be variously selected according to a subject, and preferably, the active ingredient is included in the pharmaceutical composition at a concentration of 0.01 to 5,000 μg/ml. When the concentration thereof is less than 0.01 μg/ml, pharmaceutical activity may not be exhibited, and when the concentration thereof is more than 5,000 μg/ml, toxicity to the human body may be exhibited.

The “rheumatoid arthritis” of the present disclosure is an autoimmune disease in which the immune system transmits incorrect signals and attacks healthy tissues of the body, particularly joints. Inflammation in the joints occurs, and the joints may be damaged and lose the function over time.

According to one embodiment of the present disclosure, the composition may reduce joint or cartilage inflammation.

According to one embodiment of the present disclosure, the composition may inhibit joint or cartilage damage.

According to one embodiment of the present disclosure, the composition may reduce the amount of autoantibody.

According to one embodiment of the present disclosure, the autoantibody may be CII-IgG.

The “CII IgG antibody” of the present disclosure is an antibody related to collagen type II, and is mainly related to rheumatoid arthritis (RA) and autoimmune diseases. The CII is one of main proteins that constitute the articular cartilage, which supports the joints and facilitates movement, and the CII IgG antibody may bind to the cartilage to cause inflammation and cause joint damage. The CII IgG antibody may be measured in a blood test and may be useful in diagnosing autoimmune diseases and assessing disease activity.

Further, the present disclosure provides a food composition for preventing or alleviating rheumatoid arthritis including the compound represented by Chemical Formula 1 or the pharmaceutically acceptable salt thereof as an active ingredient.

As used in the present disclosure, the “alleviation” means all actions that at least reduce parameters associated with conditions to be treated, for example, the severity of symptoms.

In addition to containing the active ingredient of the present disclosure, the food composition of the present disclosure may contain various flavoring agents, natural carbohydrates, or the like as an additional ingredient, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The food capable of adding the composition of the present disclosure includes, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohols, a carbonic acid agent used in a carbonated drink, and the like, in addition to the extract as the active ingredient. In addition, the food composition of the present disclosure may contain natural fruit juice, and pulps for preparing fruit juice beverages, and vegetable beverages.

Hereinafter, Examples of the present disclosure will be described in more detail with reference to the accompanying drawings. However, the following Examples are only intended to embody the contents of the present disclosure, and the present disclosure is not limited thereto.

### <Example 1> Preparation of Phenethyl Cinnamide Derivative Compound

### 1-1. N-phenethylcinnamamide (Compound 1)

As shown in the following Reaction Scheme 1, 370.4 mg (2.5 mmol) of cinnamic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 560 mg (1.85 eq) of 2-(4-methoxyphenyl) 2-phenylethan-1-amine and 1.5 mL (3.5 eq) of N,N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

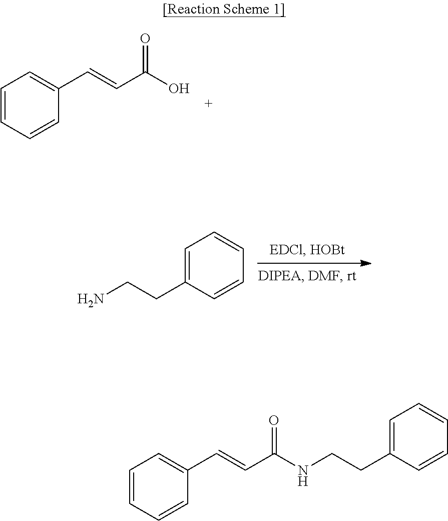 [SMILES: O=C(O)/C=C/c1ccccc1 ; NCCc1ccccc1 ; O=C(/C=C/c1ccccc1)NCCc1ccccc1]

Yield: 325 mg (52.0%).

¹H NMR (400 MHz, CDCl₃): δ 7.62 (d, J=15.6 Hz, 1H), 7.50-7.47 (m, 2H), 7.37-7.34 (m, 3H), 7.34-7.31 (m, 2H), 7.25-7.21 (m, 2H), 6.31 (d, J=15.6 Hz, 1H), 5.60 (s, 1H), 3.67 (q, J=6.6 Hz, 2H), 2.90 (t, J=6.9 Hz, 2H).

### 1-2. N-(4-methoxyphenethyl)cinnamamide (Compound 2)

As shown in the following Reaction Scheme 2, 370.4 mg (2.5 mmol) of cinnamic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 2-(4-methoxyphenyl) 2-phenylethan-1-amine and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

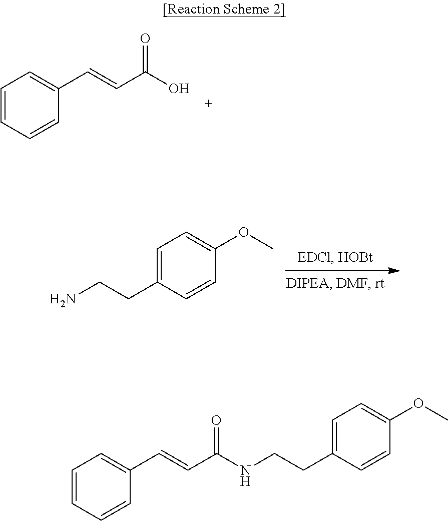 [SMILES: O=C(O)/C=C/c1ccccc1 ; COc1ccc(CCN)cc1 ; COc1ccc(CCNC(=O)/C=C/c2ccccc2)cc1]

Yield: 370 mg (63.0%).

¹H NMR (400 MHz, CDCl₃): δ 7.62 (d, J=15.6 Hz, 1H), 7.48 (dd, J=2.2 Hz, 7.3 Hz, 2H), 7.35 (q, J=2.4 Hz, 3H), 7.15 (td, J=2.5 Hz, 9.4 Hz, 2H), 6.87 (td, J=2.5 Hz, 9.4 Hz, 2H), 6.31 (d, J=15.6 Hz, 1H), 5.61 (s, 1H), 3.80 (s, 3H), 3.63 (q, J=6.5 Hz, 2H), 2.84 (t, J=6.8 Hz, 2H).

### 1-3. (E)-N-(4-methoxyphenethyl)-3-(4-methoxyphenyl)acrylamide (Compound 3)

As shown in the following Reaction Scheme 3, 445.47 mg (2.5 mmol) of (E)-3-(4-methoxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 2-(4-methoxyphenyl) 2-phenylethan-1-amine and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

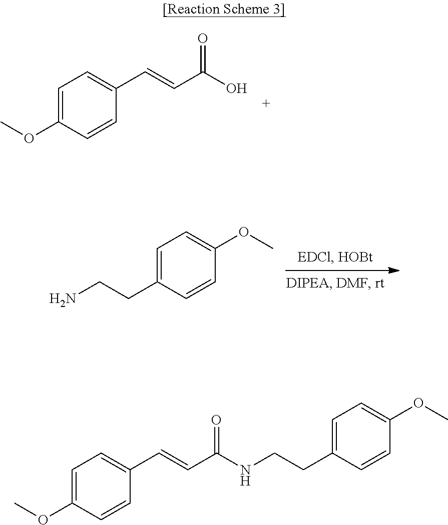

Yield: 387 mg (50.0%).

¹H NMR (400 MHz, methanol-d4): δ 7.49 (m, 7.5267-7.4676, 3H), 7.17 (d, J=8.48 Hz, 2H), 6.96 (d, J=8.80 Hz, 2H), 6.87 (dd, J=6.68 Hz, 1.96 Hz, 2H), 6.45 (d, J=15.77 Hz, 1H) 3.84 (s, J=0.00 Hz, 3H), 3.78 (s, J=0.0 Hz, 3H), 3.50 (t, J=0.00 Hz, 2H), 2.81 (t, J=7.36 Hz, 2H).

### 1-4. (E)-N-(4-hydroxyphenethyl)-3-(4-methoxyphenyl)acrylamide (Compound 4)

As shown in the following Reaction Scheme 4, 445.47 mg (2.5 mmol) of (E)-3-(4-methoxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 634.5 mg (1.85 eq) of 4-(2-aminoethyl) phenol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

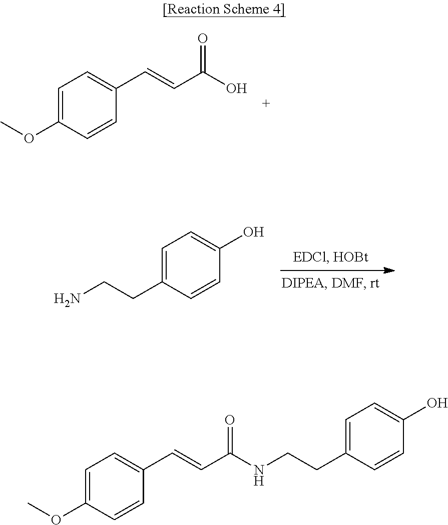 [SMILES: COc1ccc(/C=C/C(=O)O)cc1 ; NCCc1ccc(O)cc1 ; COc1ccc(/C=C/C(=O)NCCc2ccc(O)cc2)cc1]

Yield: 329 mg (44.0%).

¹H NMR (400 MHz, methanol-d4): δ 7.49 (m, 7.5194-7.4672, 3H), 7.07 (d, J=8.44 Hz, 2H), 6.95 (d, J=8.12 Hz, 2H), 6.74 (dd, J=6.48 Hz, 2.04 Hz, 2H), 6.45 (d, J=15.73 Hz, 2H), 3.84 (d, J=0.96 Hz, 1H), 3.48 (t, J=7.38 Hz, 3H), 3.33 (q, J=1.60 Hz, 2H), 2.77 (t, J=7.38 Hz, 3H).

### 1-5. (E)-N-(3,4-dihydroxyphenethyl)-3-(4-methoxyphenyl)acrylamide (Compound 5)

As shown in the following Reaction Scheme 5, 445.47 mg (2.5 mmol) of (E)-3-(4-methoxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 4-(2-aminoethyl)benzene-1,2-diol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

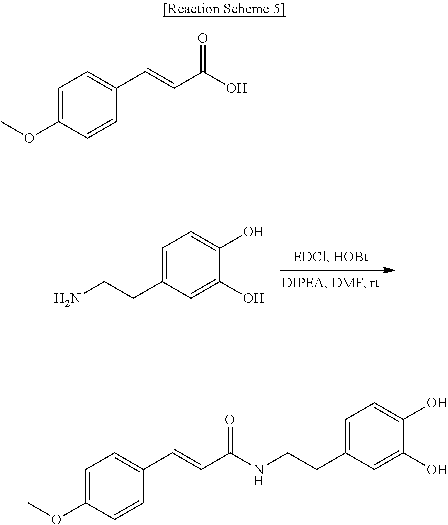 [SMILES: COc1ccc(/C=C/C(=O)O)cc1 ; NCCc1ccc(O)c(O)c1 ; COc1ccc(/C=C/C(=O)NCCc2ccc(O)c(O)c2)cc1]

Yield: 398 mg (51.0%).

¹H NMR (400 MHz, methanol-d4): δ 7.39 (d, J=15.65 Hz, 1H), 7.17 (d, J=8.72 Hz, 2H), 7.01 (d, J=2.08 Hz, 1H), 6.91 (dd, J=8.44 Hz, 2.12 Hz, 1H), 6.87 (dd, J=6.56 Hz, 2.12 Hz, 2H), 6.78 (d, J=8.16 Hz, 1H), 6.35 (d, J=15.68 Hz, 1H), 3.49 (t, J=7.38 Hz, 2H), 2.80 (t, J=7.36 Hz, 2H).

### 1-6. (E)-N-(3,4-dihydroxyphenethyl)-3-(4-methoxyphenyl)acrylamide (Compound 6)

As shown in the following Reaction Scheme 6, 410.4 mg (2.5 mmol) of (E)-3-(4-hydroxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 560 mg (1.85 eq) of 2-phenylethan-1-amine and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 ml, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

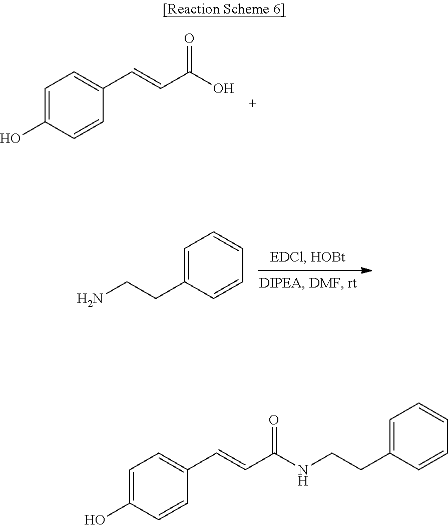 [SMILES: O=C(O)/C=C/c1ccc(O)cc1 ; NCCc1ccccc1 ; O=C(/C=C/c1ccc(O)cc1)NCCc1ccccc1]

Yield: 354 mg (53.0%).

¹H NMR (400 MHz, CDCl₃): δ 7.55 (d, J=15.6 Hz, 1H), 7.37-7.30 (m, 4H), 7.24-7.20 (m, 2H), 6.84 (d, J=8.5 Hz, 2H), 6.17 (d, J=15.6 Hz, 1H), 5.67 (d, J=5.0 Hz, 1H), 3.66 (dd, J=6.8 Hz, 12.9 Hz, 2H), 2.89 (t, J=6.8 Hz, 2H).

### 1-7. (E)-3-(4-hydroxyphenyl)-N-(4-methoxyphenethyl)acrylamide (Compound 7)

As shown in the following Reaction Scheme 7, 410.4 mg (2.5 mmol) of (E)-3-(4-hydroxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 2-(4-methoxyphenyl) 2-phenylethan-1-amine and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

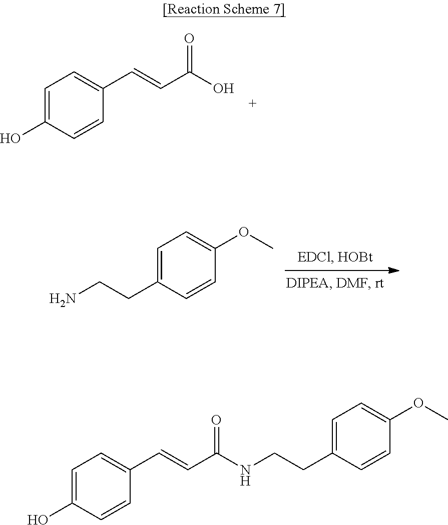 [SMILES: O=C(O)/C=C/c1ccc(O)cc1 ; COc1ccc(CCN)cc1 ; COc1ccc(CCNC(=O)/C=C/c2ccc(O)cc2)cc1]

Yield: 356 mg (46.0%).

¹H NMR (400 MHz, methanol-d4): δ 7.46 (d, J=15.77 Hz, 1H), 7.41 (dd, J=6.68 Hz, 1.88 Hz, 2H), 7.17 (dd, J=6.52 Hz, 2.08 Hz, 2H), 6.87 (dd, J=6.6 Hz, 2.04 Hz, 2H), 6.81 (dd, J=6.68 Hz, 1.96 Hz, 2H), 6.40 (d, J=15.69 Hz, 1H), 3.78 (s, J=0.0 Hz, 3H), 3.49 (t, J=7.36 Hz, 2H), 2.80 (t, J=7.34 Hz, 2H).

### 1-8. (E)-3-(4-hydroxyphenyl)-N-(4-methoxyphenethyl)acrylamide (Compound 8)

As shown in the following Reaction Scheme 8, 410.4 mg (2.5 mmol) of (E)-3-(4-hydroxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 634.5 mg (1.85 eq) of 4-(2-aminoethyl) phenol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

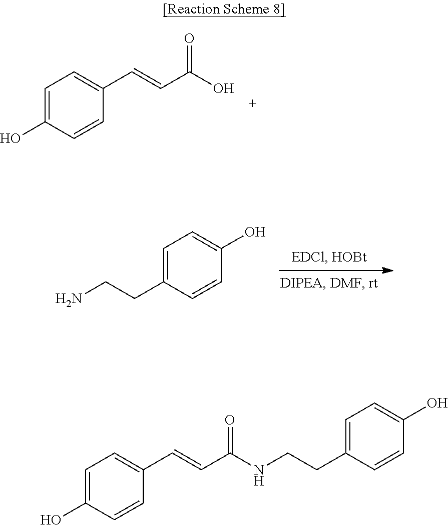 [SMILES: O=C(O)/C=C/c1ccc(O)cc1 ; NCCc1ccc(O)cc1 ; O=C(/C=C/c1ccc(O)cc1)NCCc1ccc(O)cc1]

Yield: 321 mg (45.0%).

¹H NMR (400 MHz, methanol-d4): δ 7.44 (d, J=15.74 Hz, 1H), 7.39 (d, J=8.56 Hz, 2H), 7.05 (d, J=8.56 Hz, 2H), 6.78 (d, J=8.56 Hz, 2H), 6.71 (d, J=8.56 Hz, 2H), 6.38 (d, J=15.70 Hz, 1H), 3.45 (t, J=7.40 Hz, 2H), 2.75 (t, J=7.39 Hz, 2H).

### 1-9. (E)-N-(3,4-dihydroxyphenethyl)-3-(4-hydroxyphenyl)acrylamide (Compound 9)

As shown in the following Reaction Scheme 9, 410.4 mg (2.5 mmol) of (E)-3-(4-hydroxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 4-(2-aminoethyl)benzene-1,2-diol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

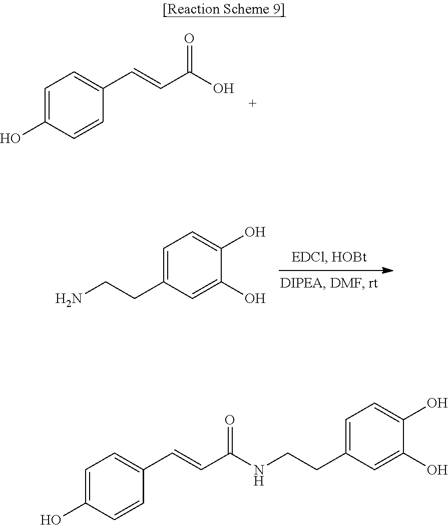 [SMILES: O=C(O)/C=C/c1ccc(O)cc1 ; NCCc1ccc(O)c(O)c1 ; O=C(/C=C/c1ccc(O)cc1)NCCc1ccc(O)c(O)c1]

Yield: 256 mg (34.0%).

¹H NMR (400 MHz, methanol-d4): δ 7.46 (d, J=15.76 Hz, 1H), 7.41 (dd, J=6.76 Hz, 1.8 Hz, 2H), 6.80 (dd, J=6.68 Hz, 1.88 Hz, 2H), 6.71 (d, J=8 Hz, 1H), 6.69 (d, J=2.04 Hz, 1H), 6.57 (dd, J=8.00 Hz, 2.08 Hz, 1H), 6.40 (d, J=15.72 Hz, 1H), 3.47 (t, J=7.38 Hz, 2H), 2.71 (t, J=7.36 Hz, 2H).

### 1-10. (2E)-3-(4-hydroxy-3-methoxyphenyl)-N-[2-(4-methoxyphenyl)ethyl]-2-propenamide (Compound 10; KMC-0245)

As shown in the following Reaction Scheme 10, 485 mg (2.5 mmol) of trans-ferulic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699 mg (1.85 eq) of 2-(4-methoxyphenyl)ethylamine and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound KMC-0245.

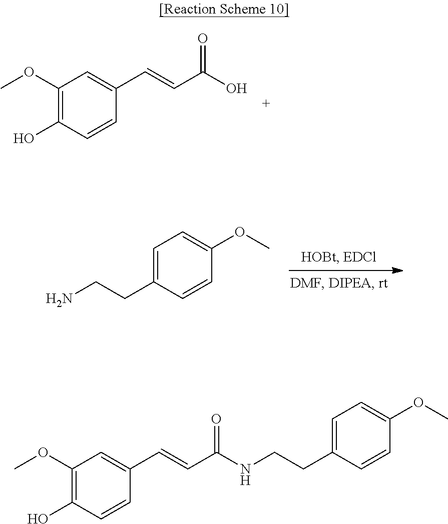

Yield: 255 mg (31.0%).

¹H NMR (400 MHz, CDCl₃): δ 7.53 (d, J=15.4 Hz, 1H), 7.14 (d, J=8.5 Hz, 2H), 7.03 (dd, J=8.1 Hz, 1.8 Hz, 1H), 6.96 (d, J=1.8 Hz, 1H), 6.89 (d, J=8.1 Hz, 1H), 6.86 (d, J=8.5 Hz, 2H), 6.17 (d, J=15.4 Hz, 1H), 5.91 (bs, 1H), 5.59 (t, J=5.1 Hz, 1H), 3.90 (s, 3H), 3.80 (s, 3H), 3.62 (q, J=6.87 Hz, 2H), 2.83 (t, J=6.8 Hz, 2H).

### 1-11. (E)-3-(4-hydroxy-3-methoxyphenyl)-N-(4-hydroxyphenethyl)acrylamide (Compound 11)

As shown in the following Reaction Scheme 11, 485 mg (2.5 mmol) of trans-ferulic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 4-(2-aminoethyl) phenol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 ml, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

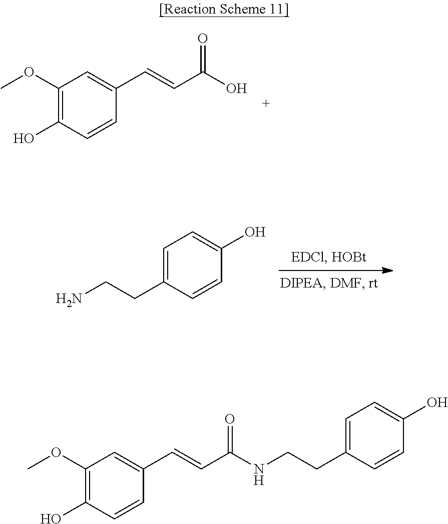 [SMILES: COc1cc(/C=C/C(=O)O)ccc1O ; NCCc1ccc(O)cc1 ; COc1cc(/C=C/C(=O)NCCc2ccc(O)cc2)ccc1O]

Yield: 256 mg (35.0%).

¹H NMR (400 MHz, methanol-d4): δ 7.45 (d, J=15.68 Hz, 1H), 7.14 (d, J=1.91 Hz, 1H), 7.08 (d, J=8.56 Hz, 2H), 7.04 (dd, J=8.44 Hz, 2.04 Hz, 1H), 6.81 (d, J=8.12 Hz, 1H), 6.74 (d, J=8.56 Hz, 2H), 6.42 (d, J=15.68 Hz, 1H), 3.90 (s, 3H), 3.48 (t, J=7.37 Hz, 2H), 2.77 (t, J=7.37 Hz, 2H).

### 1-12. (2E)-3-(4-hydroxy-3-methoxyphenyl)-N-[2-(4-methoxyphenyl)ethyl]-2-propenamide (Compound 12)

As shown in the following Reaction Scheme 12, 485 mg (2.5 mmol) of trans-ferulic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 4-(2-aminoethyl)benzene-1,2-diol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 ml, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

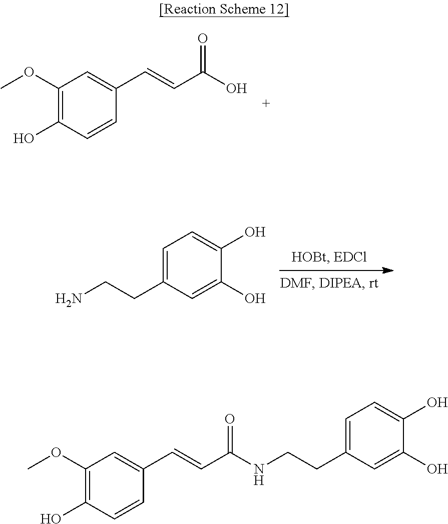 [SMILES: COc1cc(/C=C/C(=O)O)ccc1O ; NCCc1ccc(O)c(O)c1 ; COc1cc(/C=C/C(=O)NCCc2ccc(O)c(O)c2)ccc1O]

Yield: 256 mg (35.0%).

¹H NMR (400 MHz, methanol-d4): δ 7.45 (d, J=15.68 Hz, 1H), 7.14 (d, J=1.91 Hz, 1H), 7.08 (d, J=8.56 Hz, 2H), 7.04 (dd, J=8.44, 2.04 Hz, 1H), 6.81 (d, J=8.12 Hz, 1H), 6.74 (d, J=8.56 Hz, 2H), 6.42 (d, J=15.68 Hz, 1H), 3.90 (s, 3H), 3.48 (t, J=7.37 Hz, 2H), 2.77 (t, J=7.37 Hz, 2H).

### 1-13. (E)-3-(4-hydroxy-3-methoxyphenyl)-N-(4-methoxyphenethyl)acrylamide (Compound 13)

As shown in the following Reaction Scheme 13, 485 mg (2.5 mmol) of (E)-3-(3,4-dihydroxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699 mg (1.85 eq) of 2-(4-methoxyphenyl)ethylamine and 1.5 mL (3.5 eq) of N,N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

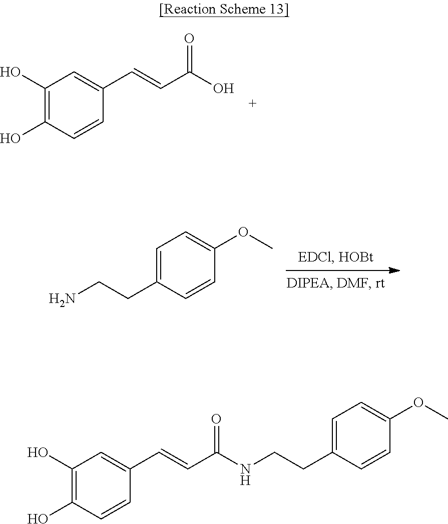 [SMILES: O=C(O)/C=C/c1ccc(O)c(O)c1 ; COc1ccc(CCN)cc1 ; COc1ccc(CCNC(=O)/C=C/c2ccc(O)c(O)c2)cc1]

Yield: 275 mg (34.0%).

¹H NMR (400 MHz, methanol-d4): δ 7.45 (d, J=15.68 Hz, 1H), 7.14 (d, J=1.91 Hz, 1H), 7.08 (d, J=8.56 Hz, 2H), 7.04 (dd, J=8.44 Hz, 2.04 Hz, 1H), 6.81 (d, J=8.12 Hz, 1H), 6.74 (d, J=8.56 Hz, 2H), 6.42 (d, J=15.68 Hz, 1H), 3.90 (s, 3H), 3.48 (t, J=7.37 Hz, 2H), 2.77 (t, J=7.37 Hz, 2H).

### 1-14. (E)-3-(3,4-dihydroxyphenyl)-N-(4-hydroxyphenethyl)acrylamide (Compound 14)

As shown in the following Reaction Scheme 14, 485 mg (2.5 mmol) of (E)-3-(3,4-dihydroxyphenyl) acrylic acid was dissolved in 16 mL of N, N-dimethylformamide (DMF, 0.15 M), and then added with 910 mg (1.1 eq) of 1-ethyl-3-(3′-dimethylaminopropyl)carbodiimide·HCl (EDC-HCl) and 675 mg (1.1 eq) of hydroxybenzotriazole (HOBt) and stirred at room temperature for 30 minutes. The mixture was added with 699.4 mg (1.85 eq) of 4-(2-aminoethyl) phenol and 1.5 mL (3.5 eq) of N, N-diisopropylethylamine (DIPEA) and stirred at room temperature for 24 hours. After the reaction was completed, the mixture was added with ultrapure water, an organic layer was extracted with ethyl acetate (10 mL, 3 times), dried over MgSO₄, and then the mixture was filtered and concentrated under reduced pressure. The concentrate was purified by silica gel column chromatography (methanol:dichloromethane=1:15, v/v) to obtain a white powdery compound.

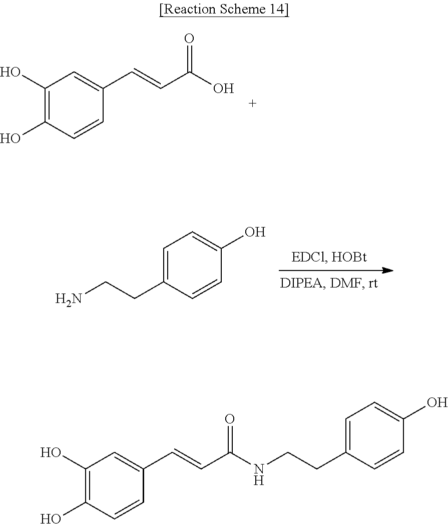 [SMILES: O=C(O)/C=C/c1ccc(O)c(O)c1 ; NCCc1ccc(O)cc1 ; O=C(/C=C/c1ccc(O)c(O)c1)NCCc1ccc(O)cc1]

Yield: 230 mg (31.0%).

¹H NMR (400 MHz, methanol-d4): δ 7.37 (d, J=15.69 Hz, 1H), 7.05 (d, J=8.52 Hz, 2H), 6.99 (d, J=1.88 Hz, 1H), 6.89 (dd, J=8.20 Hz, 1.92 Hz, 1H), 6.75 (d, J=8.12 Hz, 1H), 6.71 (dd, J=6.52 Hz, 4.92 Hz, 2H), 6.33 (d, J=15.66 Hz, 1H), 3.45 (t, J=7.40 Hz, 2H), 2.74 (t, J=7.40 Hz, 2H).

### <Example 2> Selection of Candidates

### 2-1. MTT Assay

RAW 264.7 cells were dispensed at 20×10⁴ cells/well in a 48-well plate using a DMEM medium containing 10% FBS and 1% penicillin/streptomycin, and incubated in an incubator at 37° C. and 5% CO₂ for 24 hours. Compounds 1 to 14 (80 mM) were diluted in DMSO and the medium to concentrations of 0, 0.1, 1, 10, 50, and 100 μM, and then incubated in an incubator at 37° C. and 5% CO₂ for 24 hours. The culture medium was treated with an MTT solution and further incubated for 1 hour to elute formazan crystals, and then the absorbance was measured at 560 nm using a microplate reader to calculate the cell viability. A cytotoxicity concentration was evaluated as a point at which the cell viability was reduced to 80% or less.

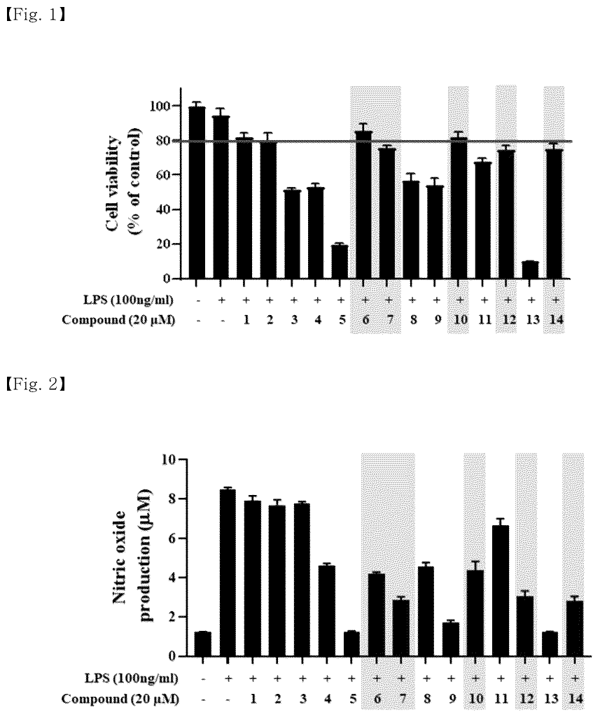

As a result, as shown in FIG. 1, it was confirmed that the cell viability was 80% or higher when treated with Compound 6, 7, 10, 12, or 14.

### 2-2. Nitric Oxide Production Inhibition Activity Assay

RAW 264.7 cells were dispensed at a density of 2×10⁵ cells/well in a 24-well plate and incubated for 24 hours. The cells were treated with diluted Compounds 1 to 14 (20 μM), left for 2 hours, and then treated with LPS at a concentration of 100 ng/ml and incubated for 18 hours to induce inflammatory responses. 100 μL of the culture medium and 100 μL of a Grease reagent were treated in a 96-well plate, reacted at room temperature for 15 minutes, and then the absorbance was measured at 540 nm to measure the level of NO present in the cell culture medium. The NO production was calculated by a standard calibration curve using a nitrite standard solution.

As a result, as shown in FIG. 2, it was confirmed that the intracellular nitric oxide production was reduced to less than 5 μM in Compounds 4 to 10 and 12 to 14.

### 2-3. TNF-α Expression Inhibitory Activity Assay

RAW 264.7 cells were dispensed at a density of 2×10⁵ cells/well in a 24-well plate and incubated for 24 hours. The cells were treated with diluted Compounds 1 to 14 (20 μM), left for 2 hours, and then treated with LPS at a concentration of 100 ng/ml and incubated for 18 hours to induce inflammatory responses. A TNF-α capture antibody was added to a 96-well plate and treated for 24 hours, and the culture medium, a TNF-α detection antibody, and streptavidin-HRP were reacted sequentially, and then added with a substrate solution to develop color, and then the reaction was stopped by adding a stop solution.

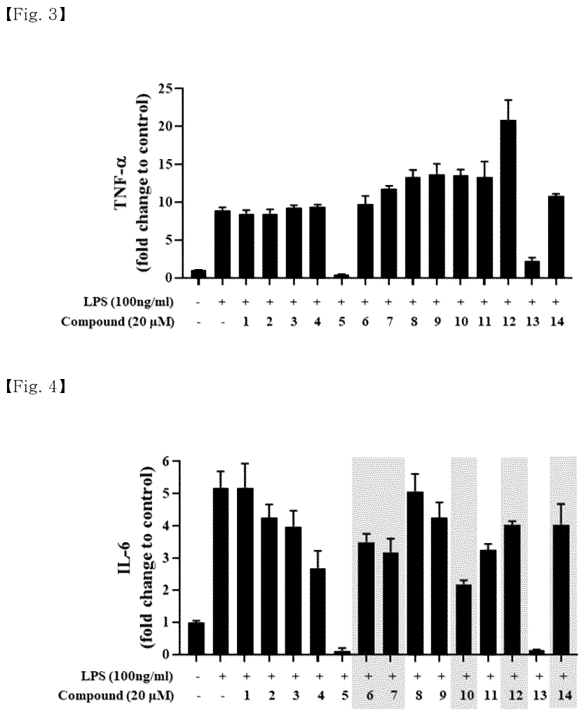

As a result, as shown in FIG. 3, it was confirmed that the TNF-α expression was completely inhibited in Compounds 5 and 13, and shown similarly to that of a control group in Compounds 1 to 4.

### 2-4. IL-6 Expression Inhibitory Activity Assay

RAW 264.7 cells were dispensed at a density of 2×10⁵ cells/well in a 24-well plate and incubated for 24 hours. The cells were treated with diluted Compounds 1 to 14 (20 μM), left for 2 hours, and then treated with LPS at a concentration of 100 ng/mL and incubated for 18 hours to induce inflammatory responses. A IL-6 capture antibody was added to a 96-well plate and treated for 24 hours, and the culture medium, a IL-6 detection antibody, and streptavidin-HRP were reacted sequentially, and then added with a substrate solution to develop color, and then the reaction was stopped by adding a stop solution.

As a result, as shown in FIG. 4, it was confirmed that Compounds 5 and 13 completely inhibited IL-6 expression, and Compounds 4 and 10 exhibited excellent IL-6 expression inhibition activity.

Therefore, among Compounds 1 to 14, Compound 10, which had the cell viability of 80% or higher and the best IL-6 expression inhibitory activity, was selected as a candidate for a rheumatoid arthritis therapeutic agent.

### <Example 3> In Vivo Rheumatoid Arthritis Efficacy

### 3-1. Rheumatoid Arthritis Clinical Scores

The efficacy of Compound 10 (KMC-0245; experimental group) represented by the following Chemical Formula 1 and Compound (KMC-0032; control group) represented by Chemical Formula 2 was evaluated in a collagen-induced arthritis (CIA) mouse model.

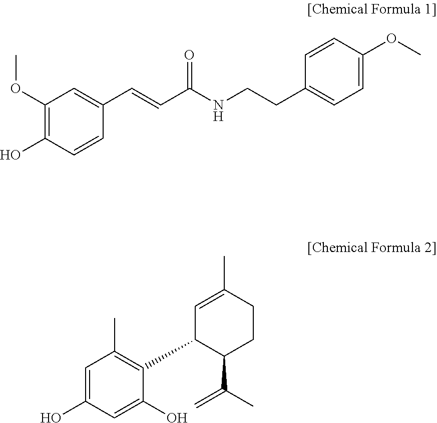 [SMILES: COc1ccc(CCNC(=O)/C=C/c2ccc(O)c(OC)c2)cc1 ; C=C(C)[C@@H]1CCC(C)=C[C@H]1c1c(C)cc(O)cc1O]

Specifically, as shown in Table 1 below, after a 7-day acclimatization period, mice (DBA1/J, Male) were sorted into groups of N mice with similar average body weights and then labeled. After one week and two weeks, 50 ML of a mixture containing 2 mg/mL of Type II collagen and CFA at a 1:1 weight ratio was injected into the tail of each mouse to perform primary and secondary immunization, and inflammatory changes in the paw tissue were recorded with clinical scores. To induce rheumatoid arthritis, 50 μg/mouse of LPS was injected intraperitoneally into mice, and a phenethyl cinnamide derivative compound KMC-0245 or a control drug MTX was administered intraperitoneally three times a week for three weeks from one week after LPS administration, and the rheumatoid arthritis clinical index was measured. The clinical index criteria for rheumatoid arthritis were shown in Table 2 below.

TABLE 1

| | Group | N | Dose (mg/kg) | Dosage&Route |
|---|---|---|---|---|
| 1 | Normal | 3 | — | — |
| 2 | CIA + Vehicle | 4 | — | 3 times/week |
| 3 | CIA + KMC-0032 | 6 | 20 | i.p. |
| 4 | CIA + KMC-0245 | 8 | 20 | |
| 5 | CIA + MTX | 4 | 5 | |

TABLE 2

| Score | Evaluation Criteria |
|---|---|
| 0 point | No swelling or edema. |
| 1 point | Mild swelling and redness only in paw or ankle joint. |
| 2 point | Mild swelling and redness from ankle joint to |
| | Metatarsal bone. |
| 3 point | Moderate swelling and redness from ankle joint |
| | to Metatarsal bone. |
| 4 point | Swelling and redness from ankle to entire leg. |

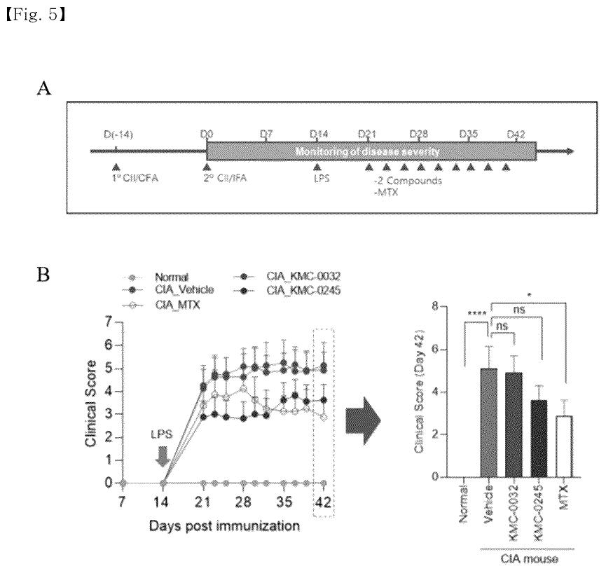

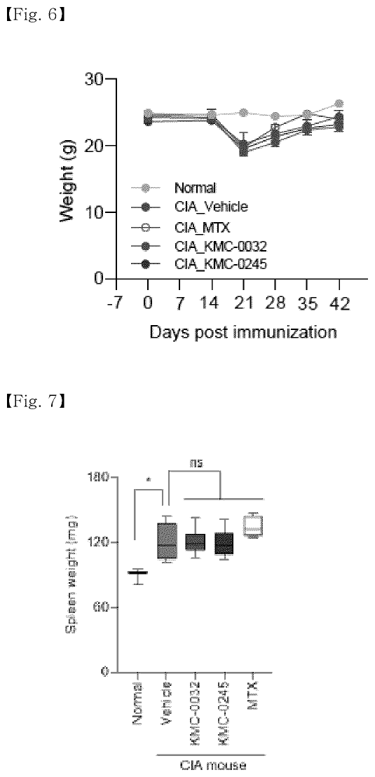

As a result, as shown in FIG. 5, through the clinical score results after 6 weeks of LPS administration, it was confirmed that the inflammation level in a Compound 10 (KMC-0245) treated group was reduced similarly to a control drug MTX treated group.

### 3-2. Weight Change Analysis

Mouse weight changes for 6 weeks according to a drug administration group were measured, and one week after the final drug administration, the mice were sacrificed, the spleens were extracted, and stored in a 4° C. Dulbecco's Phosphate-Buffered Saline (DPBS) solution, and then the spleen weights were measured.

TABLE 3
Body Weight (g)

| Group | # | DAY0 | DAY14 | DAY21 | DAY28 | DAY35 | DAY42 |
|---|---|---|---|---|---|---|---|
| Normal | 1 | 24.8 | 24.6 | 24.4 | 24.4 | 24.9 | 26.1 |
| | 2 | 25.1 | 25.2 | 26.0 | 24.9 | 24.6 | 26.6 |
| | 3 | 24.9 | 24.1 | 24.6 | 24.1 | 24.4 | 26.4 |
| CIA_Vehicle | 1 | 23.6 | 24.8 | 20.2 | 22.2 | 23.2 | 24.6 |
| | 2 | 24.5 | 23.8 | 17.6 | 19.1 | 20.6 | 23.4 |
| | 3 | 25.9 | 25.4 | 25.2 | 25.5 | 25.6 | 27.1 |
| | 4 | 22.9 | 22.6 | 18.1 | 20.4 | 22.5 | 22.3 |
| CIA_KMC-0032 | 1 | 23.4 | 23.5 | 19.2 | 21.2 | 22.2 | 22.1 |
| | 2 | 24.2 | 24.3 | 18.3 | 18.8 | 20.1 | 20.0 |
| | 3 | 26.0 | 26.5 | 20.8 | 23.1 | 24.4 | 24.4 |
| | 4 | 23.9 | 23.6 | 18.5 | 20.2 | 22.4 | 23.9 |
| | 5 | 24.0 | 23.4 | 18.8 | 18.3 | 20.6 | 21.9 |
| | 6 | 25.1 | 24.4 | 18.3 | 21.5 | 24.4 | 24.5 |
| CIA_KMC-0245 | 1 | 21.7 | 22.5 | 18.2 | 19.9 | 20.9 | 21.6 |
| | 2 | 22.1 | 22.8 | 16.5 | 19.5 | 21.3 | 22.2 |
| | 3 | 23.3 | 23.1 | 17.7 | 20.6 | 21.8 | 22.4 |
| | 4 | 23.9 | 24.7 | 17.8 | 22.1 | 23.0 | 24.4 |
| | 5 | 24.2 | 23.6 | 23.0 | 23.5 | 23.7 | 25.1 |
| | 6 | 24.7 | 24.7 | 23.1 | 23.8 | 23.7 | 23.3 |
| | 7 | 24.6 | 24.2 | 17.6 | 18.8 | 21.8 | 22.2 |
| | 8 | 24.6 | 25.0 | 22.3 | 22.9 | 24.7 | 24.6 |
| CIA_MTX | 1 | 23.4 | 22.4 | 18.9 | 22.3 | 24.6 | 23.7 |
| | 2 | 23.6 | 24.8 | 19.2 | 22.8 | 25.3 | 23.7 |
| | 3 | 25.5 | 25.7 | 20.0 | 22.9 | 24.4 | 23.6 |
| | 4 | 26.1 | 25.9 | 20.8 | 23.1 | 24.9 | 25.0 |

TABLE 4

| Group | Subject No. | Spleen Weight (mg) |
|---|---|---|
| Normal | G1-1 | 81 |
| | G1-2 | 95 |
| | G1-3 | 92 |
| CIA_Vehicle | G2-1 | 144 |
| | G2-2 | 113 |
| | G2-3 | 102 |
| | G2-4 | 121 |
| CIA_KMC-0032 | G3-1 | 142 |
| | G3-2 | 115 |
| | G3-3 | 106 |
| | G3-4 | 121 |
| | G3-5 | 116 |
| | G3-6 | 123 |
| CIA_KMC-0245 | G4-1 | 105 |
| | G4-2 | 116 |
| | G4-3 | 125 |
| | G4-4 | 130 |
| | G4-5 | 118 |
| | G4-6 | 141 |
| | G4-7 | 107 |
| | G4-8 | 113 |
| CIA_MTX | G5-1 | 147 |
| | G5-2 | 128 |
| | G5-3 | 136 |
| | G5-4 | 125 |

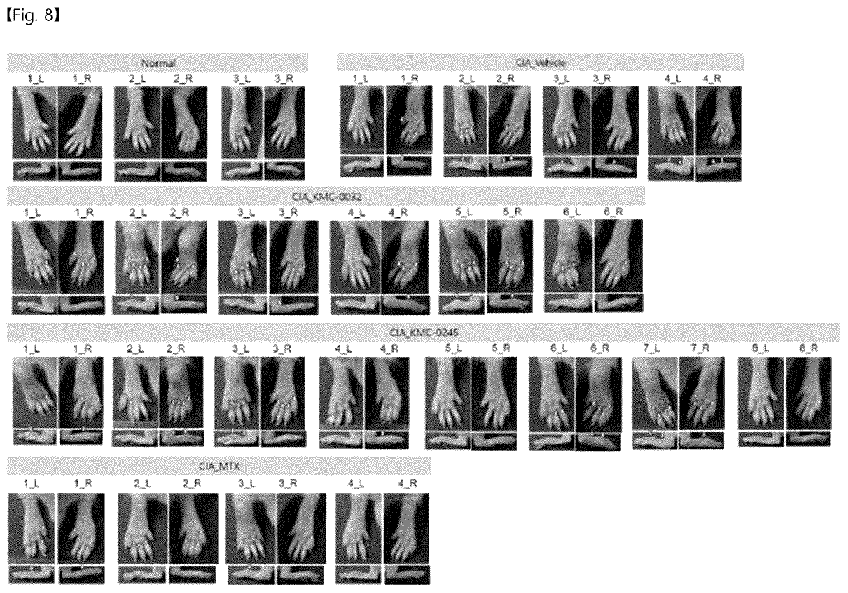

As a result, as shown in FIGS. 6 and 7, and Tables 3 and 4 above, the average body weights of the mice in all drug administration groups were decreased 3 weeks after LPS injection, but after 6 weeks, the body weights were restored similarly to the initial body weights, and the spleen weights of the rheumatoid arthritis-induced mice increased, whereas the spleen weight of each administration group remained constant.

### <Example 4> Ex Vivo Rheumatoid Arthritis Efficacy

### 4-1. Paw Tissue H&E Staining

To determine the degree of inflammation in a rheumatoid arthritis mouse model, mouse paws (marked in yellow) and H&E-stained paw tissue slides were photographed at 10× magnification using an optical microscope.

Specifically, the mouse paw tissues were fixed in a 4% paraformaldehyde (PFA) solution to remove bone minerals, and the tissues were fixed in paraffin. The paraffin tissues were cut into 4 μm-thick sections using a microtome to prepare slides, and then stained with Haematoxylin & Eosin (H&E) using an auto stainer.

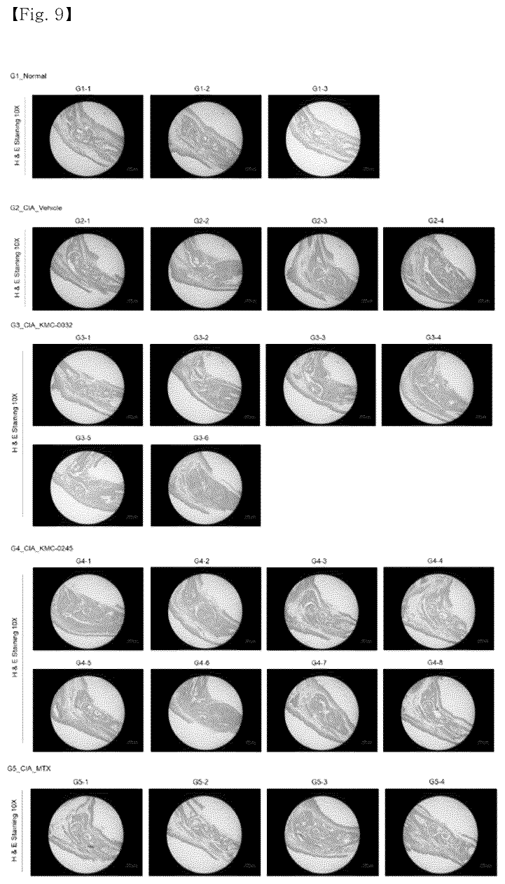

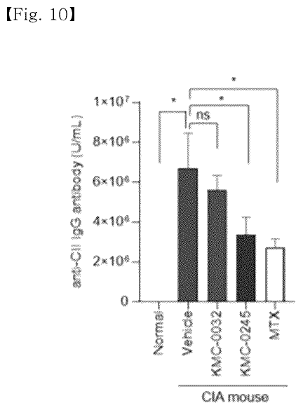

As a result, as shown in FIGS. 8 and 9, the degree of swelling of paw tissues and the number of immune cells migrating into the tissues were increased in G2-2, G2-3, and G2-4 of a CIA_Vehicle group induced with rheumatoid arthritis, tissue changes were observed in G4-1, G4-2, G4-6, and G4-7 of a CIA_KMC-0245 group, and tissue changes were observed only in G5-3 of a CIA_MTX group.

Therefore, it may be confirmed that the Compound 10 (KMC-0245) treated group inhibits changes in mouse paw tissue caused by rheumatoid arthritis similarly to the control drug MTX group.

### 4-2. CII IgG Antibody Analysis

One week after the final drug administration to the rheumatoid arthritis mouse model, the mice were sacrificed, and the bloods were separated and then analyzed for CII IgG antibody formation using an ELISA method.

Specifically, the blood was collected from the orbits of the mice anesthetized with 2.5% avertin, left at room temperature for 3 hours, and then the whole blood was centrifuged at 1, 500 rpm at 4° C. for 10 minutes to separate the serum. 1 μL of the serum was diluted in 10 mL of ELISA Buffer and treated in a Type II Collagen-coated plate well, reacted at room temperature for 2 hours, washed four times with 300 μL of Wash Buffer, and then added with an Anti-Mouse IgG-HRP Conjugate and reacted for 1 hour. The reactant was washed again with 300 μL of Wash Buffer, added with a TMB substrate, reacted for 15 minutes, and then the reaction was terminated with an HRP Stop Solution, and the O.D. values were measured at 450 nm to analyze the concentration of each sample.

TABLE 5

| Group | No. | anti-CII IgG (U/mL) |
|---|---|---|
| Normal | G1-1 | 0 |
| | G1-2 | 0 |
| | G1-3 | 0 |
| CIA_Vehicle | G2-1 | 3.5.E+06 |
| | G2-2 | 9.7.E+06 |
| | G2-3 | 8.6.E+05 |
| | G2-4 | 6.8.E+06 |
| CIA_KMC-0032 | G3-1 | 8.0.E+06 |
| | G3-2 | 4.8.E+06 |
| | G3-3 | 2.9.E+06 |
| | G3-4 | 5.0.E+06 |
| | G3-5 | 7.0.E+06 |
| | G3-6 | 5.9.E+06 |
| CIA_KMC-0245 | G4-1 | 8.1.E+06 |
| | G4-2 | 4.7.E+06 |
| | G4-3 | 5.2.E+06 |
| | G4-4 | 1.2.E+06 |
| | G4-5 | 4.5.E+05 |
| | G4-6 | 2.9.E+06 |
| | G4-7 | 2.1.E+06 |
| | G4-8 | 2.2.E+06 |
| CIA_MTX | G5-1 | 3.2.E+06 |
| | G5-2 | 1.5.E+06 |
| | G5-3 | 3.3.E+06 |
| | G5-4 | 2.9.E+06 |

As a result, as shown in FIG. 10 and Table 5 above, it was confirmed that the amount of CII IgG antibody in the serum of each subject was increased in a CIA_Vehicle group compared to a normal group, and the amount of antibody was decreased statistically significantly in a MTX group used as a control drug.

In addition, it was confirmed that in a CIA_KMC-0245 group treated with the compound KMC-0245, the CII IgG antibody was decreased to 3.4E+06 U/mL compared to 7.0E+06 U/mL in the CIA-Vehicle group, and decreased similarly to that in the control drug MTX group.

Therefore, it was confirmed that the phenethyl cinnamide derivative compound of the present disclosure had the effects of reducing an in vivo rheumatoid arthritis clinical index in a mouse model induced with rheumatoid arthritis, inhibiting damage to articular tissue, and reducing the CII IgG antibody production in mouse serum.

As described above, the specific examples of the present disclosure have been described, but those skilled in the art understanding the spirit of the present disclosure will be able to easily propose other degenerate inventions or other examples included in the scope of the present disclosure by adding, changing, and deleting other elements within the same technical scope. Therefore, it should be appreciated that the examples described above are illustrative in all aspects and are not restricted. The scope of the present disclosure is represented by claims to be described below rather than the detailed description, and it is to be interpreted that the meaning and scope of the claims and all the changes or modified forms derived from the equivalents thereof come within the scope of the present disclosure.

What is claimed is:

1. A compound represented by the following Chemical Formula 1 or a pharmaceutically acceptable salt thereof:

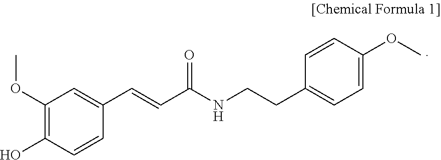 [SMILES: COc1ccc(CCNC(=O)/C=C/c2ccc(O)c(OC)c2)cc1]

2. The compound or the pharmaceutically acceptable salt thereof of claim 1, wherein the compound is a Hemp-derived phenethyl cinnamamide derivative.

3. The compound or the pharmaceutically acceptable salt thereof of claim 1, wherein the compound has cell viability of 80% or higher.

4. The compound or the pharmaceutically acceptable salt thereof of claim 1, wherein the compound inhibits nitric oxide production.

5. The compound or the pharmaceutically acceptable salt thereof of claim 1, wherein the compound inhibits IL-6 expression.

6. A pharmaceutical composition for preventing or treating rheumatoid arthritis, comprising a compound represented by the following Chemical Formula 1 or a pharmaceutically acceptable salt thereof as an active ingredient

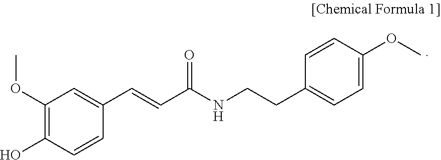 [SMILES: COc1ccc(CCNC(=O)/C=C/c2ccc(O)c(OC)c2)cc1]

7. The pharmaceutical composition of claim 6, wherein the composition reduces joint or cartilage inflammation.

8. The pharmaceutical composition of claim 6, wherein the composition inhibits joint or cartilage inflammation.

9. The pharmaceutical composition of claim 6, wherein the composition reduces the amount of autoantibody.

10. The pharmaceutical composition of claim 9, wherein the autoantibody is CII-IgG.

11. A food composition for preventing or alleviating rheumatoid arthritis, comprising a compound represented by the following Chemical Formula 1 or a pharmaceutically acceptable salt thereof as an active ingredient

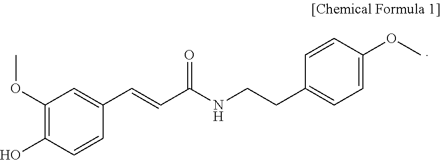 [SMILES: COc1ccc(CCNC(=O)/C=C/c2ccc(O)c(OC)c2)cc1]

12. A method for preventing or treating rheumatoid arthritis, the method comprising: administering to a subject in need thereof the compound according to claim 1 or the pharmaceutically acceptable salt thereof.
